(19) United States
(12) Patent Application Publication
(10) Pub. No.: US 2022/0208311 A1
(43) Pub. Date: Jun. 30, 2022

(54) SYSTEM FOR DETERMINING FEATURE OF ACRYLONITRILE BUTADIENE STYRENE USING ARTIFICIAL INTELLECTUAL AND OPERATION METHOD THEREOF

(71) Applicant: DAEJIN ADVANCED MATERIALS Inc., Suwon-si (KR)

(72) Inventor: Gwan Yeong KIM, Suwon-si (KR)

(73) Assignee: DAEJIN ADVANCED MATERIALS Inc., Suwon-si (KR)

(21) Appl. No.: 17/562,589

(22) Filed: Dec. 27, 2021

(30) Foreign Application Priority Data
Dec. 30, 2020 (KR) 1020200187051

(51) Int. Cl. G16C 20/70; G16C 20/30

(52) U.S. Cl. CPC G16C 20/70; G16C 20/30

(57) ABSTRACT

A system for estimating a property of a mixed material including acrylonitrile butadiene styrene (ABS) is provided. The system includes a server for analyzing data using a machine learning model, a user terminal for receiving an input of a user and transmitting the input to the server, and a data collection unit for collecting data.

### CROSS-REFERENCE TO RELATED APPLICATIONS

This application claims priority to Korean Patent Application No. 10-2020-0187051 filed on Dec. 30, 2020, the entire contents of which are herein incorporated by reference.

This application is based on the project conducted as follows:

Project Identification Number: 200159%, KMDF_PR_1415173802

Ministry Name: Research Program Title: Development of Functional Composite Material Part for Delaying Thermal Transfer between Cell Modules

Research Project Title: Development of Material Part Technology Research Management Institution: Korea Evaluation Institute of Industrial Technology

Contribution Rate: 50/50

Host Research Institute: Daejin Advanced Materials Inc.

Research Period: Apr. 1, 2021 through Dec. 31, 2024

### TECHNICAL FIELD

The present disclosure relates to a system for estimating a property and an operating method of the same. More particularly, the present disclosure relates to a system for estimating physical properties of a mixed material on the basis of a composition ratio of the mixed material using artificial intelligence.

### BACKGROUND ART

A polymer is a type of large molecule composed of repeatedly connected monomers. In general, a large molecule obtained by chemical synthesis is referred to as a “polymer.” Polymers include acrylonitrile butadiene styrene (ABS). ABS is the abbreviation of acrylonitrile, butadiene, and styrene and is mainly made of styrene among the three ingredients.

ABS is generally easily processible, highly impact resistant, and highly heat-resistant. Compared to polyethylene, ABS shows a heat resistance of 80° C. to 93° C. and an impact resistance of 0.8 to 4.5. The “impact resistance of 4.5” is strong enough not to break even when struck with a sledgehammer. Accordingly, ABS is used as a substitute for metal in industrial products such as vehicle parts, helmets, electrical equipment parts, and spinning machine parts.

With the increasing use of ABS, ABS is causing environmental pollution, and to reduce the environmental pollution, active research is underway on a method of recycling ABS. However, recycled ABS may include impurities, or the chains of polymer may be broken in recycled ABS such that the recycled ABS may have a different characteristic than general ABS. Accordingly, to produce products using recycled ABS, it is necessary to make properties of the recycled ABS similar to those of general ABS by mixing an additional material with the recycled ABS. To derive optimal ratio for mixing an additional material with recycled ABS, experiments need to be done with a great deal of time and effort.

### SUMMARY

### Technical Solution

One aspect of the present disclosure provides an operating method of a system for estimating a property of a mixed material including recycled acrylonitrile butadiene styrene (ABS), which includes a server for analyzing data using a machine learning model, a user terminal for receiving an input of a user and transmitting the input to the server, and a data collection unit for collecting data, the operating method including performing, by the data collection unit, gel permeation chromatography (GPC) analysis on recycled ABS to acquire recycled ABS composition information including a recycled ABS-related number average molecular weight and a recycled ABS-related weight average molecular weight, transmitting, by the data collection unit, the recycled ABS composition information to the server, applying, by the server, the recycled ABS composition information to a first machine learning model to acquire an index related to a characteristic of the recycled ABS, acquiring, by the user terminal, content ratios of the recycled ABS, general ABS, carbon nanotubes (CM), carbon fiber (Cr), and recycled thermoplastic polyurethane (TPU) included in a mixed material and expected property information of the mixed material, transmitting, by the user terminal, the content ratios of the recycled ABS, the general ABS, the CNT, the CF, and the recycled TPU to the server, applying, by, the server, the index related to the characteristic of the recycled ABS and the content ratios of the recycled ABS, the general ABS, the CNT, the CF, and the recycled TPU to a second machine learning model to acquire an index related to a property of the mixed material of the recycled ABS, the general ABS, the CNT, the CF, and the recycled TPU and a reliability of the index, transmitting, by the server, the index related to the property of the mixed material and the reliability of the index to the user terminal, determining, by the user terminal, a range of the property of the mixed material corresponding to the index related to the property of the mixed material, generating, by the user terminal, a first output signal representing that it is essentially necessary to perform an experiment when the reliability of the index is lower than a first threshold reliability, and generating, by the user terminal, a second output signal representing that it is necessary to perform an additional experiment when the expected property information of the mixed material is within the range of the property of the mixed material and the reliability of the index is higher than a second threshold reliability. The first machine learning model is a model which has performed machine learning on a relationship between recycled ABS composition information and indices related to the characteristic of recycled ABS, and the second machine learning model is a model which has performed machine learning on relationships between indices related to the property of mixed materials and the index related to the characteristic of the recycled ABS and content ratios of the recycled ABS, the general ABS, the CNT, the CF, and the recycled TPU.

The operating method may include acquiring, by the server, a plurality of pieces of past recycled ABS composition information and indices related to the characteristic of a plurality of pieces of past recycled ABS each corresponding to the plurality of pieces of past recycled ABS composition information and performing, by the server, machine learning on relationships between the plurality of pieces of past recycled ABS composition information and the indices related to the characteristic of the plurality of pieces of past recycled ABS to generate the first machine learning model.

The index related to the characteristic of the recycled ABS may include information related to a degree of breakage of polymer chains made of acrylonitrile, butadiene, and styrene included in the recycled ABS.

The operating method may include acquiring, by the server, the indices related to the characteristic of the plurality of pieces of past recycled ABS, content ratios of the plurality of pieces of past recycled ABS, content ratios of a plurality of pieces of past general ABS, content ratios of a plurality of pieces of past CNT, content ratios of a plurality of pieces of past CF, content ratios of a plurality of pieces of past recycled TPU, and indices related to the property of a plurality of past mixed materials and performing, by the server, machine learning on the indices related to the characteristic of the plurality of pieces of past recycled ABS, the content ratios of the plurality of pieces of past recycled ABS, the content ratios of the plurality of pieces of past general ABS, the content ratios of the plurality of pieces of past CNT, the content ratios of the plurality of pieces of past CF, the content ratios of the plurality of pieces of past recycled TPU, and the indices related to the property of the plurality of past mixed materials to generate the second machine learning model.

The acquiring of the recycled ABS composition information may include performing GPC analysis on the recycled ABS to generate a distribution of molecular weights of molecules included in the recycled ABS, performing GPC analysis on the general ABS to generate a distribution of molecular weights of molecules included in the general ABS, and determining a similarity between the distribution of the molecular weights of the molecules included in the recycled ABS and the distribution of the molecular weights of the molecules included in the general ABS. The transmitting of the recycled ABS composition information to the server may include transmitting the similarity between the distributions to the server, and the acquiring of the index related to the characteristic of the recycled ABS may include acquiring, by the server, an index related to the characteristic of the recycled ABS on the basis of the similarity between the distributions without using the first machine learning model when the similarity between the distributions is a first threshold similarity or more.

The determining of the similarity may include determining a first molecular weight (A2) having a maximum number (A1) in the distribution of the molecular weights of the molecules included in the recycled ABS, determining a second molecular weight (B2) having a maximum number (B1) in the distribution of the molecular weights of the molecules included in the general ABS, and determining the similarity between the distributions as follows: the similarity between the distributions=1/((((A2−B2)\hat{ }2)/(A2\hat{ }A2+B2\hat{ }B2))\hat{ }1/2)+(((A1−B1)\hat{ }2)/(A1\hat{ }2+B1\hat{ }2))\hat{ }(1/2)).

The acquiring of the index related to the property of the recycled ABS on the basis of the similarity may include acquiring an index related to the characteristic of the recycled ABS to represent a lowest degree of breakage of polymer chains made of acrylonitrile, butadiene, and styrene included in the recycled ABS when the similarity between the distributions is the first threshold similarity or more.

The content ratio of the general ABS may be one or less which is a ratio of a mass of the general ABS to a mass of the recycled ABS, the content ratio of the CNT may be 1/20 or less which is a ratio of a mass of the CNT to the mass of the recycled ABS, the content ratio of the CF may be 1/5 or less which is a ratio of a mass of the CF to the mass of the recycled ABS, and the content ratio of the recycled TPU may be 2/3 or less which is a ratio of a mass of the recycled TPU to the mass of the recycled ABS.

Another aspect of the present disclosure provides an operating method of a system for estimating a property of a mixed material including recycled ABS, which includes a server for analyzing data using a machine learning model, a user terminal for receiving an input of a user and transmitting the input to the server, and a data collection unit for collecting data, the operating method including performing, by the data collection unit, GPC analysis on recycled ABS to acquire recycled ABS composition information including a recycled ABS-related number average molecular weight and a recycled ABS-related weight average molecular weight, transmitting, by the data collection unit, the recycled ABS composition information to the server, acquiring, by the server, a value obtained by dividing the recycled ABS-related weight average molecular weight by the recycled ABS-related number average molecular weight as a recycled polydispersity index, applying, by the server, the recycled ABS composition information to a first machine learning model to acquire an index related to a characteristic of the recycled ABS, when the recycled polydispersity index is a threshold index or more, acquiring, by the user terminal, content ratios of the recycled ABS, the general ABS the CNT, the CF, and recycled TPU included in a mixed material and expected property information of the mixed material, transmitting, by the user terminal, the content ratios of the recycled ABS, the general ABS, the CNT, the CF, and the recycled TPU to the server, applying, by the server, the index related to the characteristic of the recycled ABS and the content ratios of the recycled ABS, the general ABS, the CNT, the CE, and the recycled TPU to a second machine learning model to acquire an index related to a property of the mixed material of the recycled ABS, the general ABS, the CNT, the CF, and the recycled TPU, transmitting, by the server, the index related to the property of the mixed material to the user terminal, and determining, by the user terminal, a range of the property of the mixed material corresponding to the index related to the property of the mixed material. The first machine learning model is a model which has performed machine learning on a relationship between recycled ABS composition information and indices related to the property of recycled ABS, and the second machine learning model is a model which has performed machine learning on relationships between indices related to the property of mixed materials and the index related to the property of the recycled ABS and content ratios of the recycled ABS, the general ABS, the CNT, the CF, and the recycled TPU.

The operating method may include acquiring, by the server, a plurality of pieces of past recycled ABS composition information and indices related to the characteristic of a plurality of pieces of past recycled ABS each corresponding to the plurality of pieces of past recycled ABS composition information and performing, by the server, machine learning on relationships between the plurality of pieces of past recycled ABS composition information and the indices related to the characteristic of the plurality of pieces of past recycled ABS to generate the first machine learning model.

The index related to the characteristic of the recycled ABS may include information related to a degree of breakage of polymer chains made of acrylonitrile, butadiene, and styrene included in the recycled ABS.

The operating method may include acquiring, by the server, the indices related to the property of the plurality of pieces of past recycled ABS, content ratios of the plurality of pieces of past recycled ABS, content ratios of a plurality of pieces of past general ABS, content ratios of a plurality of pieces of past CNT, content ratios of a plurality of pieces of past CF, content ratios of a plurality of pieces of past recycled TPU, and indices related to the property of a plurality of past mixed materials and performing, by the server, machine learning on the indices related to the characteristic of the plurality of pieces of past recycled ABS, the content ratios of the plurality of pieces of past recycled ABS, the content ratios of the plurality of pieces of past general ABS, the content ratios of the plurality of pieces of past CNT, the content ratios of the plurality of pieces of past CF, the content ratios of the plurality of pieces of past recycled TPU, and the indices related to the property of the plurality of past mixed materials to generate the second machine learning model.

The acquiring of the recycled ABS composition information may include performing GPC analysis on the recycled ABS to generate a distribution of molecular weights of molecules included in the recycled ABS, performing GPC analysis on the general ABS to generate a distribution of molecular weights of molecules included in the general ABS, and determining a similarity between the distribution of the molecular weights of the molecules included in the recycled ABS and the distribution of the molecular weights of the molecules included in the general ABS. The transmitting of the recycled ABS composition information to the server may include transmitting the similarity between the distributions to the server, and the acquiring of the index related to the characteristic of the recycled ABS may include acquiring, by the server, an index related to the characteristic of the recycled ABS on the basis of the similarity between the distributions without using the first machine learning model when the similarity between the distributions is a first threshold similarity or more and the recycled polydispersity index is a threshold index or more.

The determining of the similarity may include determining a first molecular weight (A2) having a maximum number (A1) in the distribution of the molecular weights of the molecules included in the recycled ABS, determining a second molecular weight (B2) having a maximum number (B1) in the distribution of the molecular weights of the molecules included in the general ABS, and determining the similarity between the distributions as follows: the similarity between the distributions=1/((((A2−B2)\hat{ }2)/(A2\hat{ }A2+B2\hat{ }B2))\hat{ }1/2)+(((A1−B1)\hat{ }2)/(A1\hat{ }2+B1\hat{ }2))\hat{ }(1/2)).

The acquiring of the index related to the characteristic of the recycled ABS on the basis of the similarity may include acquiring an index related to the characteristic of the recycled ABS to represent a lowest degree of breakage of polymer chains made of acrylonitrile, butadiene, and styrene included in the recycled ABS.

The acquiring of the recycled ABS composition information may include performing GPC analysis on the general ABS to acquire a general ABS-related number average molecular weight and a general ABS-related weight average molecular weight, acquiring, by the server, a value obtained by dividing the general ABS-related weight average molecular weight by the general ABS-related number average molecular weight as a general polydispersity index, and determining, by the server, a difference value between the recycled polydispersity index and the general polydispersity index. The acquiring of the index related to the characteristic of the recycled ABS may include acquiring, by the server, an index related to the characteristic of the recycled ABS on the basis of the similarity without using the first machine learning model when an absolute value of the difference value is less than a first threshold difference value and the recycled polydispersity index is less than a threshold index.

Still another aspect of the present disclosure provides an operating method of a system for estimating a property of a mixed material including recycled ABS, which includes a server for analyzing data using a machine learning model, a user terminal for receiving an input of a user and transmitting the input to the server, and a data collection unit for collecting data, the operating method including performing, by the data collection unit, GPC analysis on recycled ABS to acquire recycled ABS composition information including a recycled ABS-related number average molecular weight and a recycled ABS-related weight average molecular weight, performing GPC analysis on general ABS to acquire general ABS composition information including a general ABS-related number average molecular weight and a general ABS-related weight average molecular weight, transmitting, by the data collection unit, the recycled ABS composition information and the general ABS composition information to the server, acquiring, by the server, a value obtained by dividing the recycled ABS-related weight average molecular weight by the recycled ABS-related number average molecular weight as a recycled polydispersity index, acquiring, by the server, a value obtained by dividing the general ABS-related weight average molecular weight by the general ABS-related number average molecular weight as a general polydispersity index, acquiring an index related to a characteristic of the recycled ABS on the basis of a value obtained by subtracting the general polydispersity index from the recycled polydispersity index, acquiring, by the user terminal, content ratios of the recycled ABS, the general ABS, the CNT, the CF, and the recycled TPU included in a mixed material and expected property information of the mixed material, transmitting, by the user terminal, the content ratios of the recycled ABS, the general ABS, the CNT, the CF, and the recycled TPU to the server, applying, by the server, the index related to the characteristic of the recycled ABS and the content ratios of the recycled ABS, the general ABS, the CNT, the CF, and the recycled TPU to a second machine learning model to acquire an index related to a property of the mixed material of the recycled ABS, the general ABS, the CNT, the CF, and the recycled TPU, transmitting, by the server, the index related to the property of the mixed material to the user terminal, and determining, by the user terminal, a range of the property of the mixed material corresponding to the index related to the property of the mixed material. The second machine learning model is a model which has performed machine learning on relationships between indices related to the property of mixed materials and the index related to the characteristic of the recycled ABS and content ratios of the recycled ABS, the general ABS, the CNT, the CF, and the recycled TPU.

The index related to the characteristic of the recycled ABS may include information related to a degree of breakage of polymer chains made of acrylonitrile, butadiene, and styrene included in the recycled ABS.

The operating method may include acquiring, by the server, indices related to the property of a plurality of pieces of past recycled ABS, content ratios of the plurality of pieces of past recycled ABS, content ratios of a plurality of pieces of past general ABS, content ratios of a plurality of pieces of past CNT, content ratios of a plurality of pieces of past CF, content ratios of a plurality of pieces of past recycled TPU, and indices related to the property of a plurality of past mixed materials and performing, by the server, machine learning on the indices related to the characteristic of the plurality of pieces of past recycled ABS, the content ratios of the plurality of pieces of past recycled ABS, the content ratios of the plurality of pieces of past general ABS, the content ratios of the plurality of pieces of past CNT, the content ratios of the plurality of pieces of past CF, the content ratios of the plurality of pieces of past recycled TPU, and the indices related to the property of the plurality of past mixed materials to generate the second machine learning model.

The acquiring of the index related to the characteristic of the recycled ABS on the basis of the subtraction result value may include selecting an index related to the characteristic of the recycled ABS and corresponding to the subtraction result value from a predetermined table, and the predetermined table may be a table in which subtraction result values correspond to indices related to the property of the recycled ABS.

The acquiring of the recycled ABS composition information may include performing GPC analysis on the recycled ABS to generate a distribution of molecular weights of molecules included in the recycled ABS, performing GPC analysis on the general ABS to generate a distribution of molecular weights of molecules included in the general ABS, and determining a similarity between the distribution of the molecular weights of the molecules included in the recycled ABS and the distribution of the molecular weights of the molecules included in the general ABS. The transmitting of the recycled ABS composition information to the server may include transmitting the similarity between the distributions to the server, and the acquiring of the index related to the characteristic of the recycled ABS may include acquiring an index related to the characteristic of the recycled ABS additionally on the basis of the similarity between the distributions.

The determining of the similarity may include determining a first molecular weight (A2) having a maximum number (A1) in the distribution of the molecular weights of the molecules included in the recycled ABS, determining a second molecular weight (B2) having a maximum number (B1) in the distribution of the molecular weights of the molecules included in the general ABS, and determining the similarity between the distributions as follows: the similarity between the distributions=1/((((A2−B2)\hat{ }2)/(A2\hat{ }A2+B2\hat{ }B2))\hat{ }1/2)+(((A1−B1)\hat{ }2)/(A1\hat{ }2+B1\hat{ }2))\hat{ }(1/2)).

The acquiring of the index related to the characteristic of the recycled ABS additionally on the basis of the similarity may include acquiring an index related to the characteristic of the recycled ABS to represent a lowest degree of breakage of polymer chains made of acrylonitrile, butadiene, and styrene included in the recycled ABS.

The content ratio of the general ABS may be one or less which is a ratio of a mass of the general ABS to a mass of the recycled ABS, the content ratio of the CNT may be 1/20 or less which is a ratio of a mass of the CNT to the mass of the recycled ABS, the content ratio of the CF may be 1/5 or less which is a ratio of a mass of the CF to the mass of the recycled ABS, and the content ratio of the recycled TPU may be 2/3 or less which is a ratio of a mass of the recycled TPU to the mass of the recycled ABS.

Yet another aspect of the present disclosure provides a program for implementing the above-described operating method of a system for estimating a property of a material, the program being recorded on a computer-readable recording medium.

### DESCRIPTION OF DRAWINGS

FIG. 1 is a block diagram illustrating a system for estimating a property of a mixed material according to an embodiment of the present disclosure.

FIG. 2 is a diagram of illustrating a server according to the embodiment of the present disclosure.

FIG. 3 is a block diagram of a server (110) according to the embodiment of the present disclosure.

FIG. 4 is a flowchart illustrating an operating method of the system for estimating a property according to the embodiment of the present disclosure.

FIG. 5 is a diagram illustrating a first machine learning model according to the embodiment of the present disclosure.

FIG. 6 is a diagram illustrating a second machine learning model according to the embodiment of the present disclosure.

FIG. 7 may be a flowchart illustrating an operating method of the system for estimating a property according to the embodiment of the present disclosure.

FIG. 8 may be a diagram illustrating an operating method of the system for estimating a property according to the embodiment of the present disclosure.

FIG. 9 is a flowchart illustrating a process of acquiring an index related to a characteristic of recycled acrylonitrile butadiene styrene (ABS) without a first machine learning model according to the embodiment of the present disclosure.

FIG. 10 is a set of diagrams illustrating a process of acquiring an index related to a characteristic of recycled ABS without a first machine learning model according to the embodiment of the present disclosure.

### DETAILED DESCRIPTION

The advantages and features of disclosed embodiments and methods of achieving them will become apparent through embodiments described in detail with reference to the accompanying drawings. However, the present disclosure is not limited to the embodiments set forth herein and can be implemented in various different forms. The embodiments are merely provided to make the present disclosure complete and fully convey the scope of the invention to those skilled in the technical field to which the present disclosure pertains.

Terms used in this specification will be briefly described, and then the disclosed embodiments will be described in detail.

As the terms used herein, currently widely used general terms are selected in consideration of the functions in the present disclosure. However, these terms may vary depending on the intentions or customs of those of ordinary skill in the art, the advent of new technologies, and the like. Also, in certain cases, some terms may be arbitrarily selected by the applicant, and in this case, the meaning will be described in detail in the corresponding description of the invention. Accordingly, the terms used herein should not be defined simply by the designation of the term but should defined on the basis of the meaning of the term and the overall content of the present disclosure.

As used herein, a singular expression includes a plural expression unless clearly specified otherwise in the context. Also, a plural expression includes a singular expression unless clearly specified otherwise in the context.

Throughout the specification, when a part is referred to as “including” a component, the part does not exclude other components and may further include other components unless stated otherwise.

As used herein, the term “unit” means a software or hardware component, and a “unit” performs certain roles. However, a “unit” is not limited to the meaning of software or hardware. A “unit” may be configured to reside in an addressable storage medium or to run on one or more processors. Accordingly, as an example, a “unit” includes components, such as software components, object-oriented software components, class components, and task components, processes, functions, attributes, procedures, subroutines, segments of program code, drivers, firmware, microcode, circuitry, data, databases, data structures, tables, arrays, and variables. Functionality provided in components and “units” may be combined into fewer components and “units” or further separated into additional components and “units.”

According to an embodiment of the present disclosure, a “unit” may be implemented as a processor or a memory. The term “processor” should be interpreted broadly to encompass a general-purpose processor, a central processing unit (CPU), a microprocessor, a digital signal processor (DSP), a controller, a microcontroller, a state machine, etc. In some environments, a “processor” may refer to an application-specific integrated circuit (ASIC), a programmable logic device (PLD), a field-programmable gate array (FPGA), etc. The term “processor” may refer to a combination of processing devices, for example, a combination of a DSP and a microprocessor, a combination of a plurality of microprocessors, a combination of one or more one or more microprocessors in conjunction with a DSP core, or a combination of other such elements.

The term “memory” should be interpreted broadly to encompass any electronic component capable of storing electronic information. The term memory may refer to various types of processor-readable media such as a random access memory (RAM), a read-only memory (ROM), a non-volatile RAM (NVRAM), a programmable ROM (PROM), a erasable PROM (EPROM), an electrically erasable PROM (EEPROM), a flash memory, a magnetic or optical data storage device, and registers. A memory is referred to as being in electronic communication with a processor when the processor can read information from and/or write information to the memory. A memory integrated with a processor is in electronic communication with the processor.

Hereinafter, embodiments will be fully described with reference to the accompanying drawings so that those of ordinary skill in the art can easily implement the embodiments. Also, to clearly illustrate the present disclosure, parts not related to the description are omitted in the drawings.

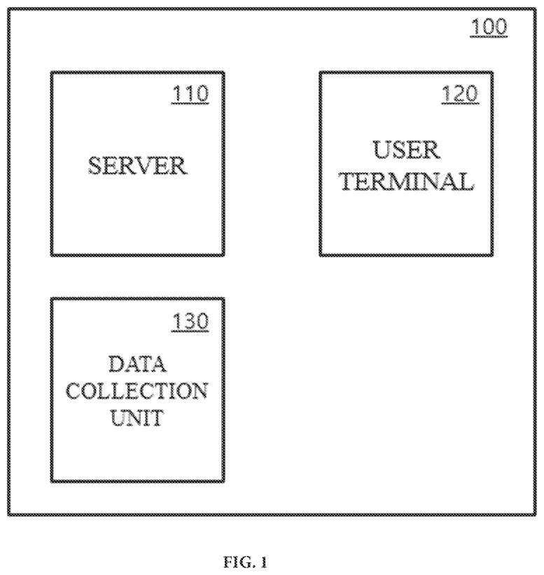

FIG. 1 is a block diagram illustrating a system for estimating a property of a mixed material according to an embodiment of the present disclosure.

A system 100 for estimating a property of a mixed material may be a system for estimating a property of a mixed material including recycled acrylonitrile butadiene styrene (ABS).

The system 100 for estimating a property of a mixed material may include a server 110 for analyzing data using a machine learning model, a user terminal 120 for receiving an input of a user and transmitting the input to the server 110, and a data collection unit 130 for collecting data.

The data collection unit 130 may receive data from an external device. Also, the data collection unit 130 may be experimental equipment for deriving a property of a mixed material. For example, the data collection unit 130 may be gel permeation chromatography (GPC) equipment.

The server 110, the user terminal 120, and the data collection unit 130 included in the system 100 will be described in further detail below.

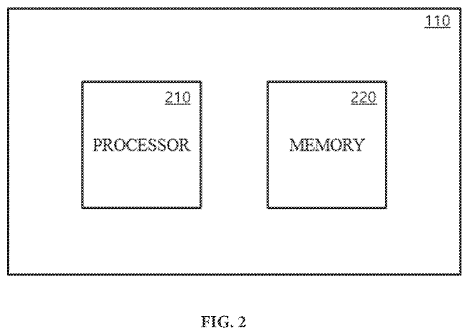

FIG. 2 is a diagram of illustrating a server according to the embodiment of the present disclosure.

The server 110 may include a processor 210 and a memory 220. The processor 210 may execute instructions stored in the memory 220.

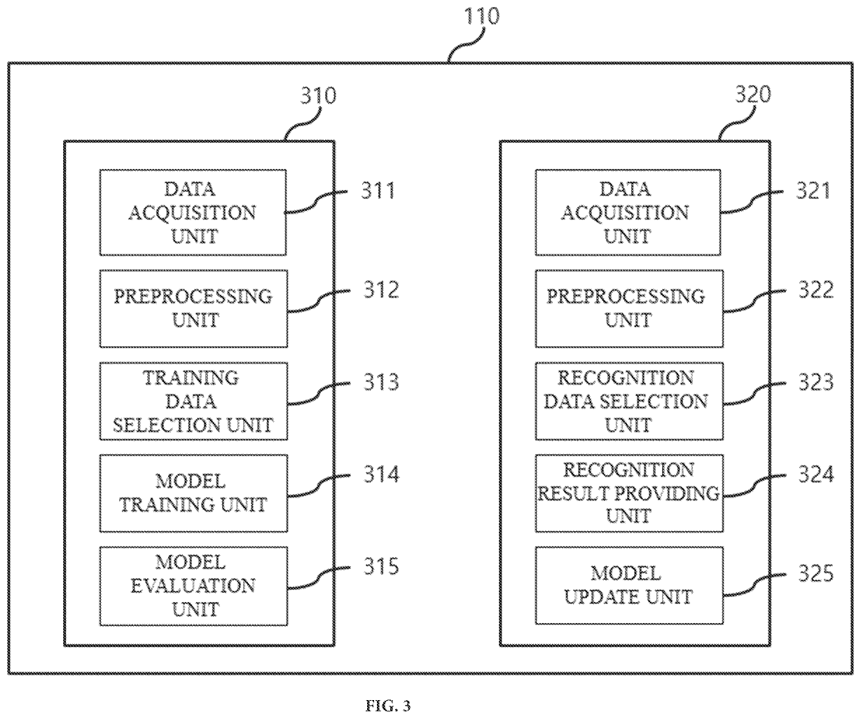

The server 110 may include a data learning unit 310 or a data recognition unit 320. The data learning unit 310 or the data recognition unit 320 will be described in detail with reference to FIG. 3. The data learning unit 310 or the data recognition unit 320 may be implemented by the processor 210 and the memory 220.

FIG. 2 illustrates the server 110 but is not limited thereto. The user terminal 120 and the data collection unit 130 of FIG. 1 may also include the processor 210 and the memory 220. Also, the server 110, the user terminal 120, and the data collection unit 130 may be connected in a wired or wireless manner and exchange data.

FIG. 3 is a block diagram of the server 110 according to the embodiment of the present disclosure.

Referring to FIG. 3, the server 110 according to the embodiment may include at least one of a data learning unit 310 and a data recognition unit 320. The above-described server 110 may include a processor and a memory.

The data learning unit 310 may train a machine learning model for performing a target task using a dataset. The data learning unit 310 may receive a dataset and label information related to the target task. The label information may be ground-truth data. The label information may be information received from a user or experimental equipment. The data learning unit 310 may acquire a machine learning model by performing machine learning on the relationship between the dataset and the label information. The machine learning model acquired by the data learning unit 310 may be a model for generating label information from a dataset.

The data recognition unit 320 may receive and store the machine learning model of the data learning unit 310. The data recognition unit 320 may output estimated label information by applying the machine learning model to input data. Also, the data recognition unit 320 may use at least one of the input data, the estimated label information, and additional data output by the machine learning model in updating the machine learning model. The additional data output by the machine learning model may be information on the reliability of the estimated label information.

At least one of the data learning unit 310 and the data recognition unit 320 may be manufactured in the form of at least one hardware chip and installed in an electronic device. For example, at least one of the data learning unit 310 and the data recognition unit 320 may be manufactured in the form of a dedicated hardware chip for artificial intelligence (A1) or manufactured as a part of an existing general-use processor (e.g., a CPU or an application processor) or a graphics processor (e.g., a graphic processing unit (GPU)) and installed in the various electronic devices described above.

Also, the data learning unit 310 and the data recognition unit 320 may be separately installed in individual electronic devices, For example, one of the data learning unit 310 and the data recognition unit 320 may be included in an electronic device, and the other may be included in the server. Also, machine learning model information built by the data learning unit 310 may be provided to the data recognition unit 320, and data input to the data recognition unit 320 may be provided to the data learning unit 310 as additional training data in a wired or wireless manner.

Meanwhile, at least one of the data learning unit 310 and the data recognition unit 320 may be implemented as a software module. When at least one of the data learning unit 310 and the data recognition unit 320 is implemented as a program including a software module (or a program module including instructions), the software module may be stored in a non-transitory computer-readable recording medium. In this case, at least one software module may be provided by an operating system (OS) or a certain application. Alternatively, a part of at least one software module may be provided by the OS, and the other part may be provided by a certain application.

The data learning unit 310 according to the embodiment of the present disclosure may include a data acquisition unit 311, a preprocessing unit 312, a training data selection unit 313, a model training unit 314, and a model evaluation unit 315.

The data acquisition unit 311 may acquire data required for machine learning. Since a great deal of data is required for learning, the data acquisition unit 311 may receive a dataset including a plurality of pieces of data.

Label information may be allocated to each of the plurality of pieces of data. The label information may be information describing each of the plurality of pieces of data. The label information may be information to be derived through the target task. The label information may be acquired from a user input, a memory, or a result of the machine learning model.

For example, the target task may be classifying characteristics of recycled ABS on the basis of recycled ABS composition information. The data acquisition unit 311 may acquire recycled ABS composition information as a plurality of pieces of data and acquire an index related to the characteristic of the recycled ABS corresponding to each piece of the recycled. ABS composition information as label information.

Also, the target task may be estimating property of a mixed material according to the content ratio of a material. In this case, the data acquisition unit 311 may acquire the content ratios of a plurality of materials as a plurality of pieces of data and acquire indices related to the property according to the content ratios of the materials as label information. The label information may be input by the user or acquired by the experimental equipment.

The preprocessing unit 312 may preprocess the acquired data so that the received data may be used for machine learning. The preprocessing unit 312 may process the acquired dataset in a preset format so that the model training unit 314 to be described below may use the dataset.

The training data selection unit 313 may select data required for learning from the preprocessed data. The selected data may be provided to the model training unit 314. The training data selection unit 313 may select data required for learning from the preprocessed data according to a preset reference. Also, the training data selection unit 313 may select data according to a reference which is preset through training by the model training unit 314 to be described below.

The model training unit 314 may learn a reference for determining which label information will be output on the basis of the dataset. Also, the model training unit 314 may perform machine learning using the dataset and label information for the dataset as training data. In addition, the model training unit 314 may perform machine learning additionally using a machine learning model which is acquired in advance. In this case, the previously acquired machine learning model may be a previously built model. For example, the machine learning model may be a model which is built in advance with received basic training data.

The machine learning model may be built in consideration of the field of application of the learning model, the purpose of learning, the computing performance of the device, etc. The machine learning model may be, for example, a neural network-based model. For example, a deep neural network (DNN), a recurrent neural network (RNN), a long short-term memory (LSTM) model, a bidirectional recurrent deep neural network (BRDNN), a convolutional neural nets network (CNN), etc. may be used as the machine learning model, but the machine learning model is not limited thereto.

According to various embodiments, when there are a plurality of previously built machine learning models, the model training unit 314 may determine a machine learning model in which there is close relation between input training data and basic training data as a machine learning model to be trained. In this case, the basic training data may be classified by data type in advance, and machine learning models may be built according to data types in advance. For example, the basic training data may be classified in advance by various references such as the place where the training data is generated, the time when the training data is generated, the size of the training data, the creator of the training data, and the type of an object in the training data.

The model training unit 314 may train the machine learning model using a training algorithm and the like including error back-propagation or gradient descent.

The model training unit 314 may train the machine learning model through, for example, supervised learning in which training data is used as input values. Also, the model training unit 314 may acquire the machine learning model through, for example, unsupervised learning in which a reference for the target task is found by learning the type of data required for the target task without supervision. Further, the model training unit 314 may acquire the machine learning model through, for example, reinforcement learning in which the feedback of whether the result of the target task obtained through learning is correct is used.

When the machine learning model is trained, the model training unit 314 may store the trained machine learning model. In this case, the model training unit 314 may store the trained machine learning model in the memory of the electronic device including the data recognition unit 320. Alternatively, the model training unit 314 may store the trained machine learning model in the memory of a server connected to the electronic device through a wired or wireless network.

The memory in which the trained machine learning model is stored may also store, for example, instructions or data related to at least one other component of the electronic device. Also, the memory may store software and/or a program. The program may include, for example, a kernel, middleware, an application programming interface (API), an application program (or “application”), and/or the like.

The model evaluation unit 315 may input evaluation data to the machine learning model and may cause the model training unit 314 to perform training again when a result output from the evaluation data does not satisfy a certain reference. In this case, the evaluation data may be preset data for evaluating the machine learning model.

For example, the model evaluation unit 315 may determine that the certain reference is not satisfied when the number or ratio of pieces of evaluation data of which recognition results are incorrect to results of the trained machine learning model with respect to the evaluation data exceeds a preset threshold value. For example, the certain reference may be defined as a ratio of 2%. In this case, when the trained machine learning model outputs incorrect recognition results with respect to more than 20 pieces of evaluation data among a total of 1000 pieces of evaluation data, the model evaluation unit 315 may evaluate the trained machine learning model to be inappropriate.

Meanwhile, when there are a plurality of trained machine learning models, the model evaluation unit 315 may evaluate whether each of the trained machine learning models satisfies the certain reference and determine a model satisfying the certain reference as a final machine learning model. In this case, when a plurality of models satisfy the certain reference, the model evaluation unit 315 may determine any one model or a certain number of models preset in order of decreasing evaluation score as final machine learning models.

Meanwhile, at least one of the data acquisition unit 311, the preprocessing unit 312, the training data selection unit 313, the model training unit 314, and the model evaluation unit 315 in the data learning unit 310 may be manufactured in the form of at least one hardware chip and installed in the electronic device. For example, at least one of the data acquisition unit 311, the preprocessing unit 312, the training data selection unit 313, the model training unit 314, and the model evaluation unit 315 may be manufactured in the form of a dedicated hardware chip for AI or may be manufactured as a part of an existing general-use processor (e.g., a CPU or an application processor) or a graphics processor (e.g., a GPU) and installed in the various electronic devices described above.

Also, the data acquisition unit 311, the preprocessing unit 312, the training data selection unit 313, the model training unit 314, and the model evaluation unit 315 may be installed in one electronic device or separately installed in individual electronic devices. For example, some of the data acquisition unit 311, the preprocessing unit 312, the training data selection unit 313, the model training unit 314, and the model evaluation unit 315 may be included in the electronic device, and the others may be included in the server.

At least one of the data acquisition unit 311, the preprocessing unit 312, the training data selection unit 313, the model training unit 314. and the model evaluation unit 315 may be implemented as a software module. When at least one of the data acquisition unit 311, the preprocessing unit 312, the training data selection unit 313, the model training unit 314, and the model evaluation unit 315 is implemented as a software module (or a program module including instructions), the software module may be stored in a non-transitory computer-readable recording medium. In this case, at least one software module may be provided by the OS or a certain application. Alternatively, a part of at least one software module may be provided by the OS, and the other part may be provided by a certain application.

The data recognition unit 320 according to the embodiment of the present disclosure may include a data acquisition unit 321, a preprocessing unit 322, a recognition data selection unit 323, a recognition result providing unit 324, and a model update unit 325.

The data acquisition unit 321 may receive input data. The preprocessing unit 322 may preprocess the acquired input data so that the acquired input data may be used by the recognition data selection unit 323 or the recognition result providing unit 324.

The recognition data selection unit 323 may select required data from the preprocessed data. The selected data may be provided to the recognition result providing unit 324. The recognition data selection unit 323 may select some or all of the preprocessed data according to a preset reference. Also, the recognition data selection unit 323 may select data according to a reference preset through training by the model training unit 314.

The recognition result providing unit 324 may apply the selected data to a machine learning model to acquire result data. The machine learning model may be the machine learning model generated by the model training unit 314. The recognition result providing unit 324 may output the result data.

The model update unit 325 may have the machine learning model updated on the basis of the recognition result provided by the recognition result providing unit 324. For example, the model update unit 325 provides the recognition result provided by the recognition result providing unit 324 to the model training unit 314 so that the model training unit 314 updates the machine learning model.

Meanwhile, at least one of the data acquisition unit 321, the preprocessing unit 322 the recognition data selection unit 323, the recognition result providing unit 324, and the model update unit 325 may be manufactured in the form of at least one hardware chip and installed in the electronic device. For example, at least one of the data acquisition unit 321, the preprocessing unit 322, the recognition data selection unit 323, the recognition result providing unit 324, and the model update unit 325 may be manufactured in the form of a dedicated hardware chip for AI or may be manufactured as a part of an existing general-use processor (e.g., a CPU or an application processor) or a graphics processor (e.g., a GPU) and installed in the various electronic devices described above.

Also, the data acquisition unit 321, the preprocessing unit 322, the recognition data selection unit 323, the recognition result providing unit 324, and the model update unit 325 may be installed in one electronic device or separately installed in individual electronic devices. For example, some of the data acquisition unit 321, the preprocessing unit 322, the recognition data selection unit 323, the recognition result providing unit 324, and the model update unit 325 may be included in the electronic device, and the others may be included in the server.

At least one of the data acquisition unit 321, the preprocessing unit 322, the recognition data selection unit 323, the recognition result providing unit 324, and the model update unit 325 may be implemented as a software module. When at least one of the data acquisition unit 321, the preprocessing unit 322, the recognition data selection unit 323, the recognition result providing unit 324, and the model update unit 325 is implemented as a software module (or a program module including instructions), the software module may be stored in a non-transitory computer-readable recording medium. In this case, at least one software module may be provided by the OS or a certain application. Alternatively, a part of at least one software module may be provided by the OS, and the other part may be provided by a certain application.

A method and device for the data acquisition unit 311, the preprocessing unit 312, and the training data selection unit 313 of the data learning unit 310 to receive and process training data will be described in further detail below.

The user terminal 120 may acquire information related to an ABS product from a sticker attached to the surface of the ABS product before recycling. The sticker attached to the surface of the ABS product may be a sticker attached by the manufacturer of the ABS product. The sticker may include information related to recycling of the ABS product. For example, the user terminal 120 may capture the image of a barcode or quick response (QR) code attached to the surface of the ABS product with a camera. Also, the user terminal 120 may acquire information related to the ABS product from the image of the barcode or QR code. The information related to the ABS product may include information on the ratios of acrylonitrile, butadiene, and styrene included in the ABS product. The user terminal 120 may transmit the information related to the ABS product to the server 110.

The server 110 may determine whether the ABS product is recyclable on the basis of the received information related to the ABS product. For example, the server 110 may acquire a first mass ratio of the mass of acrylonitrile to the mass of butadiene on the basis of the information related to the ABS product. Also, the server 110 may acquire a second mass ratio of the mass of styrene to the mass of butadiene on the basis of the information related to the ABS product. The server 110 may store a predetermined first ratio range and second ratio range. When the first mass ratio is within the first ratio range and the second mass ratio is within the second ratio range, the server 110 may determine that the ABS product is recyclable. Also, when the first mass ratio is out of the first ratio range or the second mass ratio is out of the second ratio range, the server 110 may determine that the ABS product is not recyclable. In this way, the server 110 allows recycled ABS to be acquired only using ABS having a specific composition ratio such that recycled ABS having a property desired by the user can be easily obtained.

When it is determined that the ABS product is recyclable, the server 110 may transmit the first mass ratio and the second mass ratio to the data collection unit 130. Also, the server 110 may allow analysis on recycled ABS, which is obtained from the ABS product, to be started by the data collection unit 130.

During the process of generating recycled ABS, a material other than ABS may be mixed therewith. A third mass ratio of the mass of acrylonitrile to the mass of butadiene included in the recycled ABS may be acquired by the data collection unit 130, the experimental equipment, or the user. Also, a fourth mass ratio of the mass of styrene to the mass of butadiene included in the recycled ABS may be acquired by the data collection unit 130, the experimental equipment, or the user. The data collection unit 130 may acquire the third mass ratio and the fourth mass ratio. The data collection unit 130 may determine whether the difference between the first mass ratio and the third mass ratio is a threshold mass ratio or less. Also, the data collection unit 130 may determine whether the difference between the second mass ratio and the fourth mass ratio is the threshold mass ratio or less. The threshold mass ratio may be a predetermined mass ratio. When the absolute value of the difference between the first mass ratio and the third mass ratio is the threshold mass ratio or less and the absolute value of the difference between the second mass ratio and the fourth mass ratio is the threshold mass ratio or less, the data collection unit 130 may allow other materials to be mixed with the recycled ABS such that the recycled ABS has a target property. An experiment for checking whether the target property is obtained is performed through the following process.

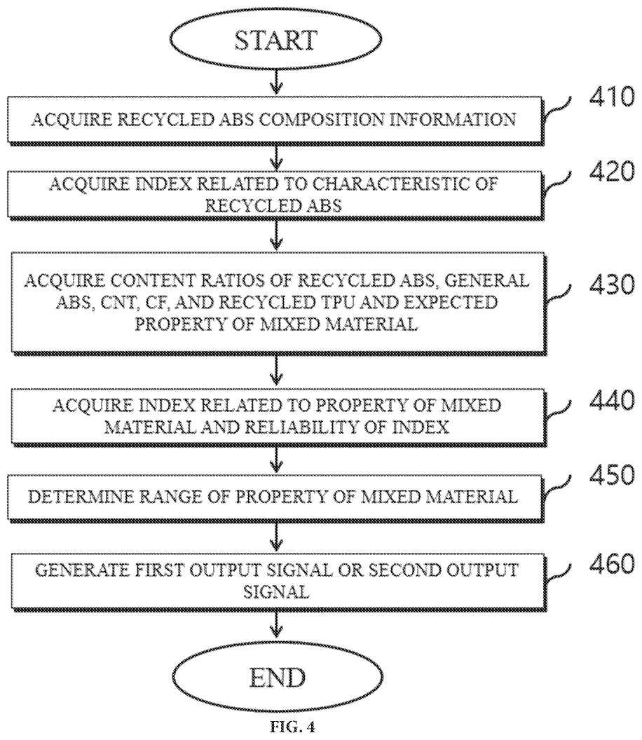

FIG. 4 is a flowchart illustrating an operating method of the system for estimating a property according to the embodiment of the present disclosure.

The data collection unit 130 may perform a step 410 of performing GPC analysis on the recycled ABS to acquire recycled ABS composition information including a recycled ABS-related number average molecular weight and a recycled ABS-related weight average molecular weight.

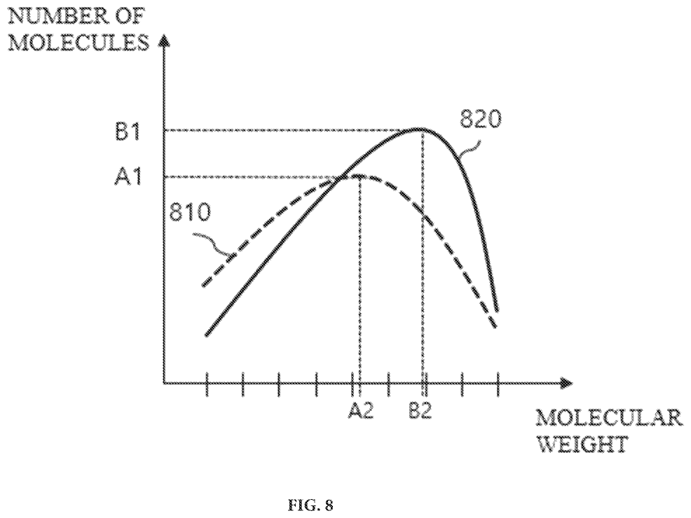

The data collection unit 130 may include a GPC analysis apparatus. The GPC analysis apparatus is an apparatus for calculating a molecular weight distribution from the calibration plots of standard materials “of which molecular weights are already known” and the chromatogram of a sample “of which molecular weight is unknown” by causing a material with a light molecular weight to be left in a column for a long time and a material with a heavy molecular weight to leak out fast using the pore size of filler particles in the column. The GPC analysis apparatus will be described in FIG. 8, but in brief, the GPC analysis apparatus releases large molecules fast and slowly releases small molecules due to resistance by the pores of filler particles.

The data collection unit 130 which is the GPC analysis apparatus may acquire information on the number of molecules with respect to the molecular weight of a polymeric material. Also, the data collection unit 130 may acquire a number average molecular weight and a weight average molecular weight on the basis of the information on the number of molecules with respect to the molecular weight. For example, the data collection unit 130 may acquire a recycled ABS-related number average molecular weight and a recycled ABS-related weight average molecular weight on the basis of information on the number of molecules with respect to the molecular weight of the recycled ABS.

In the present disclosure, recycled ABS may be ABS that has already been used once as a product. In the present disclosure, general ABS means newly produced ABS. During a recycling process, the chains of polymer in recycled ABS may be broken, and a material other than those constituting ABS may be mixed therewith. Accordingly, a recycled ABS-related number average molecular weight and a recycled ABS-related weight average molecular weight may differ from a general ABS-related number average molecular weight and a general ABS-related weight average molecular weight.

The data collection unit 130 may perform a step of transmitting the recycled ABS composition information to the server 110. The server 110 may receive the recycled ABS composition information from the data collection unit 130.

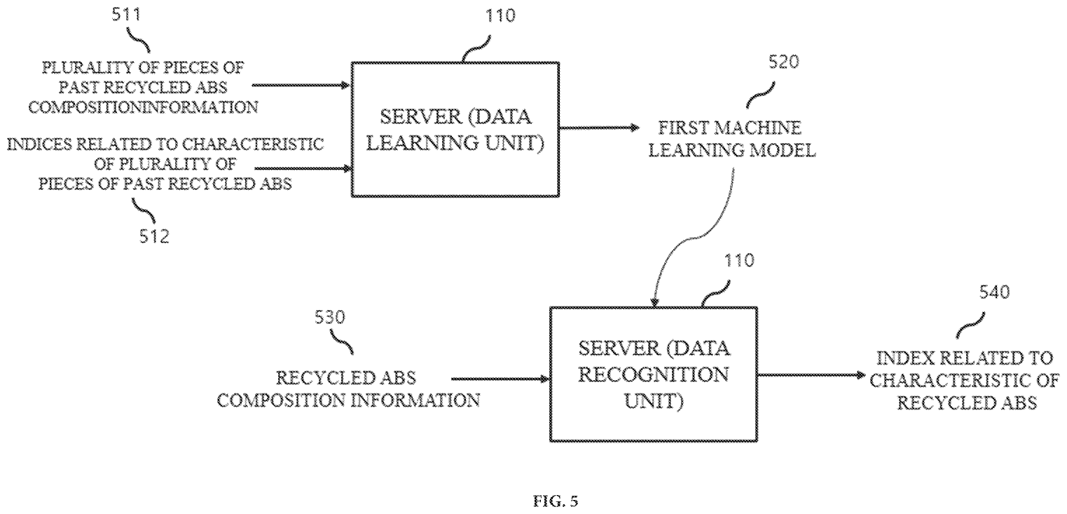

The server 110 may perform a step 420 of acquiring an index related to a characteristic of the recycled ABS by applying the recycled ABS composition information to a first machine learning model. The first machine learning model may be a machine learning model that has performed machine learning on the relationship between recycled ABS composition information and indices related to the characteristic of recycled ABS. The first machine learning model may be generated by the data learning unit 310 of the server 110. The server 110 may acquire the first machine learning model from the memory. The server 110 may receive the first machine learning model from an external device. The first machine learning model will be described in detail below with reference to FIG. 5.

The server 110 may output the index related to the characteristic of the recycled ABS through the first machine learning model. The index related to the characteristic of the recycled ABS output by the first machine learning model may be an estimated index.

The index related to the characteristic of the recycled ABS represents the degree of similarity between the recycled ABS and the general ABS. ABS includes acrylonitrile, butadiene, and styrene. The property of ABS may slightly vary depending on the ratios of acrylonitrile, butadiene, and styrene. In the present disclosure, general ABS may be ABS that has not been recycled but has been newly generated. Also, general ABS may be ABS that is a criterion for determining a characteristic of the recycled ABS. The index related to the characteristic of the recycled ABS may represent the degree of difference between the composition ratios of acrylonitrile, butadiene, and styrene of the recycled ABS and the composition ratios of the general ABS. For example, the index related to the characteristic of the recycled ABS may be the difference between styrene/acrylonitrile of the recycled ABS and styrene/acrylonitrile of the general ABS when the molecular ratio of butadiene is 1.

Also, the index related to the characteristic of the recycled ABS may include information related to the degree of breakage of polymer chains made of acrylonitrile, butadiene, and styrene included in the recycled ABS. In other words, the index related to the characteristic of the recycled ABS may represent the degree of breakage of polymer chains of the recycled ABS in comparison with the general ABS. During a recycling process, chains of the recycled ABS may be broken. Accordingly, the recycled ABS may include many molecules having a relatively lower molecular weight than the general ABS. Consequently, the index related to the characteristic of the recycled ABS may have a larger value when a greater number of chains are broken.

Unlike the step 420 of FIG. 4, the server 110 may perform a step of acquiring an index related to the characteristic of the recycled ABS without using the first machine learning model. According to various embodiments of the present disclosure, the server 110 may perform a step of acquiring a value obtained by dividing the recycled ABS-related weight average molecular weight by the recycled ABS-related value average molecular weight as a recycled polydispersity index.

A polydispersity index is a measure indicating how different the molecular weights of polymer molecules (high molecules) in a specific polymer are. The weight average molecular weight of a polymeric material is higher than the number average molecular weight. Accordingly, the polydispersity index may have a value of one or more. A polydispersity index having a greater value may mean that there is a greater difference between the molecular weights of high molecules. In the case of the general ABS which has not been recycled, high molecules constituting the general ABS have relatively uniform molecular weights. Accordingly, the polydispersity index may have a low value. In the case of the recycled ABS, impurities are mixed or chains are broken during the recycling process, and thus high molecules constituting the ABS have relatively nonuniform molecular weights. Accordingly, the polydispersity index may have a high value.

The server 110 may determine whether the recycled polydispersity index is a threshold index or more. The threshold index may be a predetermined value. The threshold index may be an experimentally acquired value. The threshold index may be a value for determining whether the recycled ABS is sufficiently uniform.

When the recycled polydispersity index is the threshold index or more, the server 110 may perform a step of acquiring an index related to the characteristic of the recycled ABS by applying the recycled ABS composition information to the first machine learning model. This may be the same as the step 420 of FIG. 4.

However, when the recycled polydispersity index is less than the threshold index, the server 110 may perform a step of acquiring an index related to the characteristic of the recycled ABS on the basis of the recycled polydispersity index without applying the recycled ABS composition information to the first machine learning model. For example, the server 110 may store a table in which recycled polydispersity indices correspond to indices related to the characteristic of the recycled ABS. The server 110 may select an index related to the characteristic of the recycled ABS from the table on the basis of the received recycled polydispersity index.

According to various embodiments of the present disclosure, the step 410 in which the data collection unit 130 acquires the recycled ABS composition information may further include the following process. The data collection unit 130 may perform a step of acquiring a general ABS-related number average molecular weight and a general ABS-related weight average molecular weight by performing GPC analysis on the general ABS. The data collection unit 130 may further transmit the general ABS-related number average molecular weight and general ABS-related weight average molecular weight to the server 110.

The server 110 may perform a step of acquiring a value obtained by dividing the recycled ABS-related weight average molecular weight by the recycled ABS-related value average molecular weight as a recycled polydispersity index, The server 110 may perform a step of acquiring a value obtained by dividing the general ABS-related weight average molecular weight by the general ABS-related value average molecular weight as a general polydispersity index. The server 110 may perform a step of determining the difference value between the recycled polydispersity index and the general polydispersity index. The difference value may be obtained by subtracting the general polydispersity index from the recycled polydispersity index.

During the step 420 of acquiring an index related to the characteristic of the recycled ABS, the server 110 may perform the following process.

The server 110 may determine whether the absolute value of the difference value is less than a first threshold difference value and whether the recycled polydispersity index is less than a threshold index, The first threshold difference value may be a predetermined value. The first threshold difference value ay be a value for determining whether there is a great difference between the recycled polydispersity index and the general polydispersity index. Also, the threshold index may be a predetermined value. The threshold index may be an experimentally acquired value. The threshold index may be a value for determining whether the recycled ABS is sufficiently uniform.

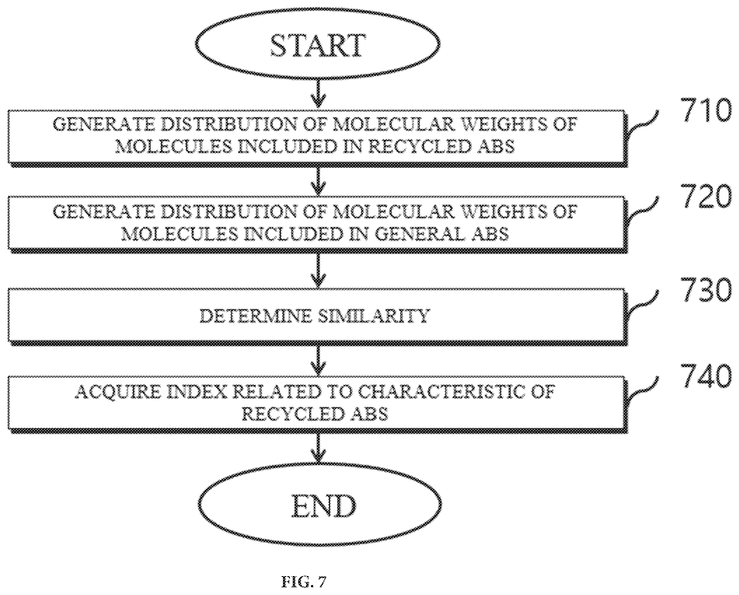

When the absolute value of the difference value is less than the first threshold difference value and the recycled polydispersity index is less than the threshold index, the server 110 may perform a step of acquiring an index related to the characteristic of the recycled ABS on the basis of similarity without using the first machine learning model. The similarity means the similarity between a distribution 810 and a distribution 820 and may be described with reference to FIGS. 7 and 8.

When the absolute value of the difference value is the first threshold difference value or more or the recycled polydispersity index is the threshold index or more, the server 110 may perform a step of acquiring an index related to the characteristic of the recycled ABS using the first machine learning model as the step 420.

Using the first machine learning model may require a relatively high processing capability. The server 110 may not use the first machine learning model under a specific condition to reduce the consumption of the processing capability while maintaining the system performance for estimating a property.

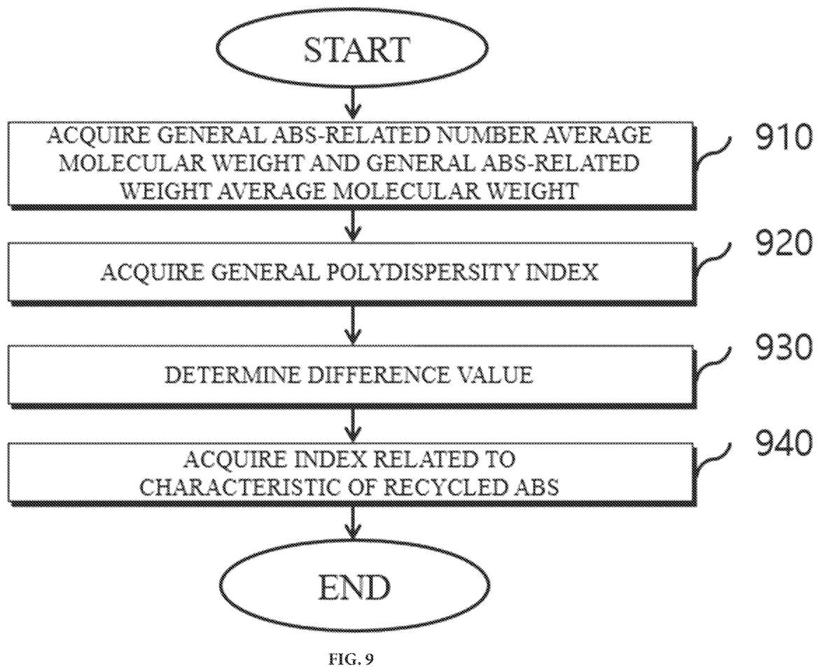

According to various embodiments of the present disclosure, the step 410 in which the data collection unit 130 acquires the recycled ABS composition information may further include the following process. This will be described with reference to FIG. 9.

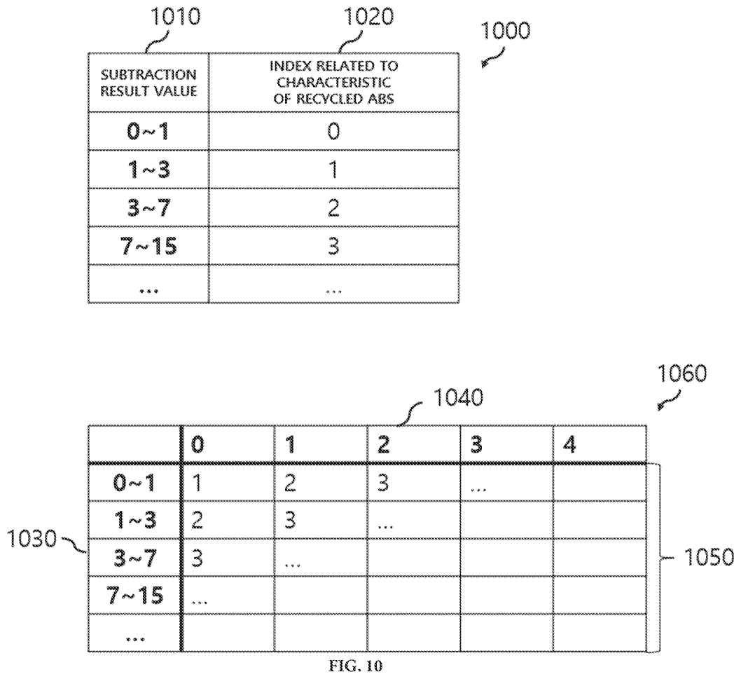

FIG. 9 is a flowchart illustrating a process of acquiring an index related to a characteristic of recycled ABS without a first machine learning model according to the embodiment of the present disclosure. FIG. 10 is a set of diagrams illustrating a process of acquiring an index related to a characteristic of recycled ABS without a first machine learning model according to the embodiment of the present disclosure.

Referring to FIG. 9, the data collection unit 130 may perform a step 910 of acquiring a general ABS-related number average molecular weight and a general ABS-related weight average molecular weight by performing GPC analysis on the general ABS. The data collection unit 130 may further transmit the general ABS-related number average molecular weight and the general ABS-related weight average molecular weight to the server 110.

The server 110 may perform a step of acquiring a value Obtained by dividing a recycled ABS-related weight average molecular weight by a recycled ABS-related number average molecular weight as a recycled polydispersity index. The server 110 may perform a step 920 of acquiring a value obtained by dividing a general ABS-related weight average molecular weight by a general ABS-related number average molecular weight as a general polydispersity index. The server 110 may perform a step 930 of determining the difference between the recycled polydispersity index and the general polydispersity index. The difference value may be obtained by subtracting the general polydispersity index from the recycled polydispersity index. Also, the step 420 of acquiring an index related to the characteristic of the recycled ABS may be replaced by the following step. The server 110 may perform a step 940 of acquiring an index related to the characteristic of the recycled ABS on the basis of the value obtained by subtracting the general polydispersity index from the recycled polydispersity index.

Referring to FIG. 10, the server 110 may store a predetermined table 1000 in which subtraction result values 1010 correspond to indices 1020 related to the characteristic of the recycled ABS. The server 110 may select the index 1020 related to the characteristic of the recycled ABS corresponding to the subtraction result value 1010 from the predetermined table 1000.

Referring to FIG. 10, the server 110 may store a predetermined table 1060 in which subtraction result values 1030 correspond to indices 1050 related to the characteristic of the recycled ABS with respect to distribution similarities 1040. The server 110 may select the index 1050 related to the characteristic of the recycled ABS and corresponding to the subtraction result value 1030 and the distribution similarity 1040 from the predetermined table 1060. The distribution similarity may be as follows.

$$Distribution similarity = 1 / ( ( ( ( A 2 - B 2 ) \hat{} 2 ) / ( A 2 \hat{} 2 + B 2 \hat{} 2 ) ) \hat{} ( 1 / 2 ) + ( ( ( A 1 - B 1 ) \hat{} 2 / ( A 1 \hat{} 2 + B 1 \hat{} 2 ) ) \hat{} ( 1 / 2 ) )$$

Briefly referring to FIG. 8, A1 is the maximum number in the distribution of molecular weights of molecules included in the recycled ABS, and in this case, the molecular weight of the recycled ABS may be a first molecular weight A2. Also, B1 is the maximum number in the distribution of molecular weights of molecules included in the general ABS, and in this case, the molecular weight of the general ABS may be a second molecular weight B2.

The distribution similarity 1040 will be described in further detail with reference to FIGS. 7 and 8.

Using the first machine learning model may require a relatively high processing capability. The server 110 may not use the first machine learning model at all to reduce the consumption of the processing capability while maintaining the accuracy of the system for estimating a property.

According to various embodiments of the present disclosure, the step 410 in which the data collection unit 130 acquires the recycled ABS composition information may further include the following process. This will be described with reference to FIG. 9.

Referring back to FIG. 4, the user terminal 120 may perform a step 430 of acquiring the content ratios of the recycled ABS, the general ABS, the CNT, the CF, and the recycled TPU included in a mixed material and expected property information of the mixed material. The recycled ABS may not have the property desired by the user. Accordingly, the user may put in effort to generate a mixed material having the desired property by mixing the recycled ABS, the general ABS, the CNT, the CT, and the recycled TPU. The recycled TPU may be replaced by general TPU which has not been recycled but has been newly generated. The CNT may be muti-wall carbon nanotube (MWCNT). The CF may be milled carbon fiber.

The user may want to know physical characteristics of a mixed material generated by mixing the recycled ABS, the general ABS, the CNT, the CE, and the recycled TPU. The user terminal 120 may receive the content ratios of the recycled ABS, the general ABS, the CNT, the CF, and the recycled TPU from the user.

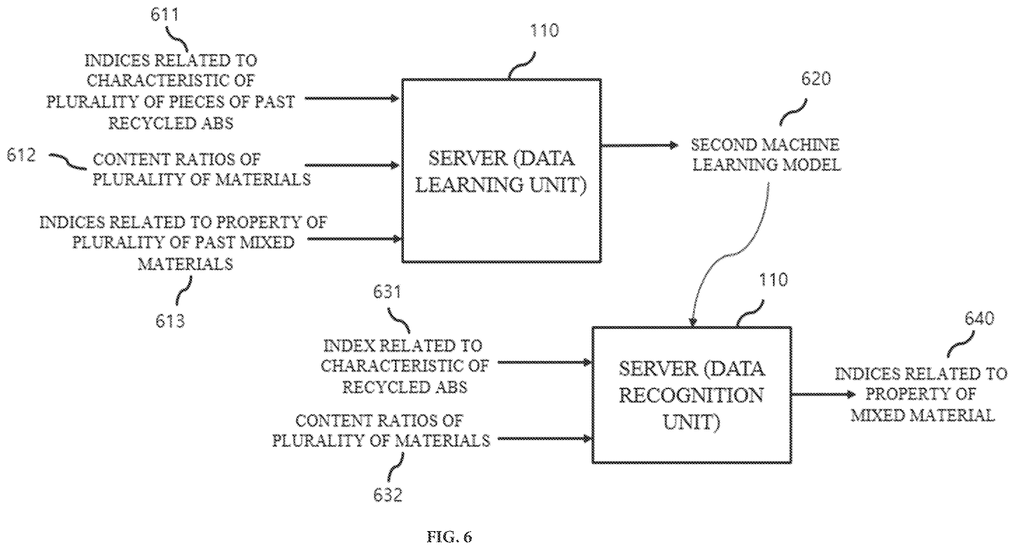

The system for estimating a property may show high accuracy within a specific range of contents. As illustrated in FIG. 6, a content ratio 612 of a plurality of material is within a specific range of content ratios, and thus the second machine learning model ensures its reliability only within the specific range of content ratios. To increase the reliability of the system for estimating a property, the user terminal 120 may limit the content ratios of the recycled ABS, the general ABS, the CNT, the CF, and the TPU. For example, as the content ratio of the general ABS, a ratio of the mass of the general ABS to the mass of the recycled ABS may be one or less. In other words, the content ratio of the general ABS/the mass of the recycled ABS may be one or less. As the content ratio of the CNT, a ratio of the mass of the CNT to the mass of the recycled ABS may be 1/20 or less. In other words, the mass of the CNT/the mass of the recycled ABS may be 1/20 or less. Also, as the content ratio of the CF, a ratio of the mass of the CF to the mass of the recycled ABS may be 1/5 or less. In other words, the mass of the CF/the mass of the recycled ABS may be 1/5 or less. Also, as the content ratio of the recycled TPU, a ratio of the mass of the recycled TPU to the mass of the recycled ABS may be 2/3 or less. In other words, the mass of the TPU/the mass of the recycled ABS may be 2/3 or less. The content ratios of the general ABS, the CNT, the CE, and the TPU may be 0 or more.

Also, the user terminal 120 may further receive the expected property information from the user. The expected property information may include information on the type of property and information on the value of the property. The information on the type of property may include at least one of tensile strength, impact strength, electrical conductivity, flexural strength, and hardness. The information on the value of the property may represent the magnitude of one of the tensile strength, the impact strength, the electrical conductivity, the flexural strength, and the hardness.

The user terminal 120 may perform a step of transmitting the content ratios of the recycled ABS, the general ABS, the CNT, the CF, and the recycled TPU to the server 110. The server 110 may receive the content ratios of the recycled ABS, the general ABS, the CNT, the CF, and the recycled TPU from the user terminal 120.

Also, the user terminal 120 may further transmit the expected property information to the server 110. The server 110 may receive the expected property information from the user terminal 120. The server 110 may select a second machine learning model on the basis of the expected property information. For example, the server 110 may select a second machine learning model corresponding to the type of property on the basis of the information on the type of property included in the expected property information. The server 110 may accurately estimate the property of the mixed material by selecting a second machine learning model specialized in the specific type of property.

CNT may be used for increasing the tensile strength or electrical conductivity of the recycles ABS. The general ABS may be used for increasing the impact strength of the recycled ABS. Also, CF may be used for increasing the electric conductivity of the recycled ABS. TPU may be used for increasing the impact strength of the recycled ABS.

The server 110 may perform a step 440 of acquiring at least one of an index related to the property of the mixed material of the recycled ABS, the general ABS, the CNT, the CF, and the recycled TPU and the reliability of the index by applying the index related to the characteristic of the recycled ABS and the content ratios of the recycled ABS, the general ABS, the CNT, the CF, and the recycled TPU to the second machine learning model.

The second machine learning model may be a model that has performed machine learning on the relationship between the index related to the characteristic of the recycled ABS and the content ratios of the recycled ABS, the general ABS, the CNT, the CF, and the recycled TPU and the index related to the property of the mixed material. The second machine learning model may be generated by the data learning unit 310 of the server 110. The server 110 may acquire the second machine learning model from the memory. The server 110 may acquire the second machine learning model from an external device. The second machine learning model will be described with reference to FIG. 6.

The server 110 may output the index related to the property of the mixed material and the reliability of the index through the second machine learning model. The index related to the property of the mixed material output through the second machine learning model may be an estimated index. The reliability of the index may be a reliability estimated by the second machine learning model. The reliability of the index may be a value related to the degree of similarity between the index related to the property of the mixed material estimated by the second machine learning model and a ground-truth index related to the property of the mixed material or a probability that the two indices are the same. The reliability of the index may be a value determined by the softmax function of the output layer of the second machine learning model.

The index related to the property may include information on the type of property and information on the range of property values. The information on the type of property may include tensile strength, impact strength, electrical conductivity, flexural strength, or hardness. The second machine learning model may output an index related to one property. However, the number of properties related to an index output by the second machine learning model is not limited thereto, and the second machine learning model may output an index related to at least one property.

The server 110 may perform a step of transmitting the index related to the property of the mixed material and the reliability of the index to the user terminal 120. The user terminal 120 may receive the index related to the property of the mixed material and the reliability of the index from the server 110.

The user terminal 120 may perform a step 450 of determining a property range of the mixed material corresponding to the index related to the property of the mixed material. As described above, the index related to the property may include the information on the range of property values. In other words, indices may correspond to the ranges of property values on a one-to-one basis. The user terminal 120 may store a table in which indices correspond to the ranges of property values. The user terminal 120 may acquire the range of property values corresponding to the index related to the property estimated by the server.

Also, the user terminal 120 may perform a step 460 of generating a first output signal and a second output signal. More specifically, when the reliability of the index is lower than a first threshold reliability, the user terminal 120 may perform a step of generating a first output signal representing that it is essentially necessary to perform an experiment. The first threshold reliability may be a predetermined value. The user terminal 120 may output the first output signal as audio or video. The user may check the output first output signal and determine to experiment on the property of the mixed material generated with the content ratios of the recycled ABS, the general ABS, the CNT, the CF, and the recycled TPU. This is because when the user terminal 120 outputs the first output signal, there is a high possibility that the estimation of the server 110 is incorrect.

Also, when the expected property of the mixed material is within the property range of the mixed material and the reliability of the index is higher than a second threshold reliability, the user terminal 120 may perform a step of generating a second output signal representing that it is necessary to perform an additional experiment. The second threshold reliability may be a predetermined value. The second threshold reliability may be greater than the first threshold reliability.

The first output signal and the second output signal may be different. The first output signal may represent that an experiment is essential for the user to check the property of the mixed material, and the second output signal may represent that it is necessary to actually check the estimated property of the mixed material through an experiment.

The user terminal 120 may output the second output signal as audio or video. The user may check the output second output signal and determine to experiment on the property of the mixed material generated with the content ratios of the recycled ABS, the general ABS, the CNT the CF, and the recycled TPU. When the user terminal 120 outputs the second output signal, the estimation of the server 110 may be correct, but it is necessary to actually check whether a mixed material is generated with the desired property through an experiment.

When neither of the above-described conditions for generating the first output signal and the second output signal is satisfied, the user terminal 120 may generate a third output signal representing that the expected property of the mixed material is not within the property range of the mixed material. Accordingly, the user may confirm the third output signal output from the user terminal 120 and may not perform an experiment. In this way, the system for estimating a property according to the present disclosure can reduce experiments to be performed by the user such that the burden of experiments can be eased.

FIG. 5 is a diagram illustrating the first machine learning model according to the embodiment of the present disclosure.

The server 110 may include the data learning unit 310. The server 110 may perform a step of acquiring a plurality of pieces of past recycled ABS composition information 511 and indices 512 related to a characteristic of a plurality of pieces of past recycled ABS each corresponding to the plurality of pieces of past recycled ABS composition information 511. The plurality of pieces of past recycled ABS composition information 511 and the indices 512 related to the characteristic of the plurality of pieces of past recycled ABS may be received from an external device or acquired from the memory. The plurality of pieces of past recycled ABS composition information 511 may correspond to the indices 512 related to the characteristic of the plurality of pieces of past recycled ABS on a one-to-one basis. The indices 512 related to the characteristic of the plurality of pieces of past recycled ABS may be ground-truth values input by a person.

The indices 512 related to the characteristic of the plurality of pieces of past recycled ABS may represent the degree of breakage of polymer chains of the recycled ABS in comparison with the general ABS. An index related to a characteristic of the recycled ABS represents the degree of similarity between the recycled ABS and the general ABS. For example, the index related to the characteristic of the recycled ABS may have a larger value when a greater number of chains are broken.

The server 110 may perform a step of generating a first machine learning model 520 by performing machine learning on the relationship between the plurality of pieces of past recycled ABS composition information 511 and the indices 512 related to the characteristic of the plurality of pieces of past recycled ABS. The process of performing machine learning on the relationship between pieces of data has been described above together with the data learning unit 310 of FIG. 3, and the description will not be reiterated.

The server 110 may store the first machine learning model 520. Also, the server 110 may receive the first machine learning model 520 from another server. The server 110 may include the data recognition unit 320. The server 110 may acquire recycled ABS composition information 530. The server 110 may perform a step 420 of acquiring an index 540 related to the characteristic of the recycled ABS by applying the recycled ABS composition information to the first machine learning model. The index 540 related to the characteristic of the recycled ABS is a value estimated by the server 110 and thus may differ from the indices 512 related to the characteristic of the plurality of pieces of past recycled ABS.

FIG. 6 is a diagram illustrating the second machine learning model according to the embodiment of the present disclosure.

The server 110 may perform a step of acquiring indices 611 related to the characteristic of a plurality of pieces of past recycled ABS, content ratios of the plurality of pieces of past recycled ABS, a plurality of pieces of past general ABS, a plurality of pieces of past CNT, a plurality of pieces of past CF, and a plurality of pieces of past recycled TPU, and indices 613 related to the property of a plurality of past mixed materials.

The server 110 may acquire the indices 611 related to the characteristic of the plurality of pieces of past recycled ABS and content ratios 612 of the plurality of materials. The content ratios of the plurality of materials may include the content ratios of the plurality of pieces of past recycled. ABS, the content ratios of the plurality of pieces of past general ABS, the content ratios of the plurality of pieces of past CNT, the content ratios of the plurality of pieces of past CF, and the content ratios of the plurality of pieces of past recycled TPU. Also, the server 110 may acquire the indices 613 related to the property of the plurality of past mixed materials. The indices 611 related to the characteristic of the plurality of pieces of past recycled ABS, the content ratios 612 of the plurality of materials, and the indices 613 related to the property of the plurality of past mixed materials may correspond to each other on a one-to-one basis. The indices related to the characteristic of the plurality of pieces of past recycled ABS may be values estimated by the first machine learning model of the server 110. In other words, the indices 611 related to the characteristic of the plurality of pieces of past recycled ABS may correspond to the index 540 related to the characteristic of the recycled ABS of FIG. 5.

The indices 613 related to the property of the plurality of past mixed materials may be ground-truth values. In other words, the user or experimental equipment may determine the indices 613 related to the property of the plurality of past mixed materials on the basis of the indices 611 related to the characteristic of the plurality of pieces of past recycled ABS and the content ratios of the plurality of pieces of past recycled ABS, the plurality of pieces of past general ABS, the plurality of pieces of past CNT, the plurality of pieces of past CF, and the plurality of pieces of past recycled TPU. The server 110 may receive the indices 613 related to the determined property of the plurality of past mixed materials from the user or experimental equipment.

The property of the plurality of past mixed materials acquired from the user or experimental equipment may be the property of a mixed material produced in a specific way. For example, for the mixed material, the recycled ABS, the general ABS, the CNT, the CF, and the TPU may be mixed and kneaded. A kneading temperature may be 190 degrees to 210 degrees. During the kneading, the load of a motor may be 1.3 A. Also, the kneaded mixture may be ground. The grinding time may be about 10 minutes. The ground mixture may be injected at 220 degrees to 240 degrees such that a final mixed material may be produced. The property of the plurality of past mixed materials may be the property of the final mixed material.

The data learning unit 310 included in the server 110 may perform a step of generating a second machine learning model 620 by performing machine learning on the relationships between the indices related to the property of the plurality of past mixed materials and the indices related to the characteristic of the plurality of pieces of past recycled ABS, the content ratios of the plurality of pieces of past recycled ABS, the content ratios of the plurality of pieces of past general ABS, the content ratios of the plurality of pieces of past CNT, the content ratios of the plurality of pieces of past CF, and the content ratios of the plurality of pieces of past recycled TPU. The process of performing machine learning on the relationship between pieces of data has been described above together with the data learning unit 310 of FIG. 3, and the description will not be reiterated.

The server 110 may store the second machine learning model 620. Also, the server 110 may receive the second machine learning model 620 from another server.

The server 110 may include the data recognition unit 320. The server 110 may acquire an index 631 related to the characteristic of the recycled ABS and content ratios 632 of the plurality of materials. The content ratios of the plurality of materials may include the content ratios of the plurality of pieces of past recycled ABS, the content ratios of the plurality of pieces of past general ABS, the content ratios of the plurality of pieces of past CNT, the content ratios of the plurality of pieces of past CF, and the content ratios of the plurality of pieces of past recycled TPU.

The server 110 may perform a step 440 of acquiring at least one of an index 640 related to the property of a mixed material of the recycled ABS, the general ABS, the CNT, the CF, and the recycled TPU and the reliability of the index by applying an index related to the characteristic of the recycled ABS and the content ratios of the recycled ABS, the general ABS, the CNT, the CF, and the recycled TPU to the second machine learning model. The index 640 related to the property of the mixed material is a value estimated by the server 110 and thus may differ from the indices 613 related to the property of the plurality of past mixed materials.

The index 640 related to the property of the mixed material may represent an estimated property of the mixed material produced in a specific way. As described above, for the mixed material, the recycled ABS, the general ABS, the CNT, the CF, and the TPU may be mixed and kneaded. A kneading temperature may be 190 degrees to 210 degrees. During the kneading, the load of a motor may be 1.3 A. Also, the kneaded mixture may be ground. The grinding time may be about 10 minutes. The ground mixture may be injected at 220 degrees to 240 degrees such that a final mixed material may be produced. The index 640 related to the property of the mixed material may be an estimated property of the final mixed material produced through the above process.

FIG. 7 may be a flowchart illustrating an operating method of the system for estimating a property according to the embodiment of the present disclosure. FIG. 8 may be a diagram illustrating an operating method of the system for estimating a property according to the embodiment of the present disclosure.

The step 410 of acquiring the recycled ABS composition information may further include the following step. The data collection unit 130 may perform a step 710 of generating the distribution of molecular weights of molecules included in the recycled ABS by performing GPC analysis on the recycled ABS. FIG. 8 shows a distribution 810 of molecular weights of molecules included in the recycled ABS. Also, the data collection unit 130 may perform a step 720 of generating the distribution of molecular weights of molecules included in the general ABS by performing GPC analysis on the general ABS. Here, the molecules may be polymer. FIG. 8 shows a distribution 820 of molecular weights of molecules included in the recycled ABS.

The data collection unit 130 may perform a step 730 of determining the similarity between the distribution 810 of molecular weights of molecules included in the recycled ABS and the distribution 820 of molecular weights of the molecules included in the general ABS. The data collection unit 130 may use various algorithms to determine the similarity between the distribution 810 and the distribution 820. In this regard, since the recycled ABS is obtained by recycling the general ABS, the following method may be used.

The recycled ABS is obtained by recycling a product that is originally the general ABS. During the process of generating the recycled ABS, the chains of polymer included in the recycled ABS may be broken. Accordingly, molecules included in the recycled ABS may have a lighter molecular weight than molecules included in the general ABS. More specifically, a first molecular weight A2 having a maximum number A1 in the distribution 810 of molecular weights of molecules included in the recycled ABS may be smaller than a second molecular weight B2 having a maximum number B1 in the distribution 820 of molecular weights of molecules included in the general ABS.

Therefore, to perform a step of determining the similarity between distributions, the data collection unit 130 may perform the following process. The data collection unit 130 may perform a step of determining the first molecular weight A2 having the maximum number A1 in the distribution of molecular weights of molecules included in the recycled ABS. The data collection unit 130 may perform a step of determining the second molecular weight B2 having the maximum number B1 in the distribution of molecular weights of molecules included in the general ABS. Also, the data collection unit 130 or the server 110 may perform the step of determining the similarity between the distributions on the basis of the following equation.

$$The similarity between the distributions = 1 / ( ( ( ( A 2 - B 2 ) \hat{} 2 ) / ( A 2 \hat{} 2 + B 2 \hat{} 2 ) ) \hat{} ( 1 / 2 ) + ( ( ( A 1 - B 1 ) \hat{} 2 ) / ( A 1 \hat{} 2 + B 1 \hat{} 2 ) ) \hat{} ( 1 / 2 ) )$$

When the data collection unit 130 transmits the recycled ABS composition information to the server 110, the data collection unit 130 may transmit the similarity between the distributions to the server 110.

When performing the step 420 of acquiring an index related to the characteristic of the recycled ABS, the server 110 may further perform the following process. When the similarity between the distributions is a first threshold similarity or more, the server 110 may perform a step 740 of acquiring an index related to the characteristic of the recycled ABS on the basis of the similarity between the distributions without using the first machine learning model. When the similarity between the distributions is the first threshold similarity or more, the server 110 can ease the processor's burden of processing by not using the first machine learning model. Also, the above equation which is used so as not to use the first machine learning model is relatively simple and thus may not put a heavy burden on many processors.

The first threshold similarity may be a predetermined value. The first threshold value may be a value for determining whether the distribution 810 of molecular weights of molecules included in the recycled ABS is similar to the distribution 820 of molecular weights of molecules included in the general ABS. In other words, when the similarity between the distributions is the first threshold value or more, the distribution 810 of molecular weights of molecules included in the recycled ABS may be similar to the distribution 820 of molecular weights of molecules included in the general ABS.

More specifically, the step 740 of acquiring an index related to the characteristic of the recycled ABS on the basis of the similarity between the distributions may include the following step. In other words, when the similarity between the distributions is the first threshold similarity or more, the server 110 may perform a step of acquiring an index related to the characteristic of the recycled ABS to represent the lowest degree of breakage of polymer chains made of acrylonitrile, butadiene, and styrene included in the recycled ABS. As described above, the index related to the characteristic of the recycled ABS may represent the degree of breakage of polymer chains of the recycled ABS in comparison with the general ABS. In other words, when the degree of breakage of polymer chains of the recycled ABS is greater in comparison with the general ABS, the index may have a larger value. Also, when the degree of breakage of polymer chains of the recycled ABS is less in comparison with the general ABS, the index may have a smaller value. When the similarity between the distributions is the first threshold similarity or more, the server 110 may determine the index related to the characteristic of the recycled ABS to be the lowest.

According to various embodiments of the present disclosure, the server 110 may further acquire a recycled polydispersity index. When the similarity between the distributions is the first threshold similarity or more and the recycled polydispersity index is the threshold index or more, the server 110 may perform a step of acquiring an index related to the characteristic of the recycled ABS on the basis of the similarity between the distributions without using the first machine learning model. For example, the server 110 may store a table in which recycled polydispersity indices correspond to indices related to the characteristic of the recycled ABS. The server 110 may select an index related to the characteristic of the recycled ABS in the table on the basis of the received recycled polydispersity index. The server 110 may not use the first machine learning model under a specific condition to reduce the consumption of the processing capability while maintaining the accuracy of the system for estimating a property.

According to various embodiments of the present disclosure, the server 110 may perform a step 940 of acquiring an index related to the characteristic of the recycled ABS on the basis of a value obtained by subtracting the general polydispersity index from the recycled polydispersity index without considering whether to use the first machine learning model and without the first machine learning model.

Referring to FIG. 10, the server 110 may store the predetermined table 1000 in which the subtraction result values 1010 correspond to the indices 1020 related to the characteristic of the recycled ABS. The server 110 may select the index 1020 related to the characteristic of the recycled ABS corresponding to the subtraction result value 1010 from the predetermined table 1000.

Referring to FIG. 10, the server 110 may store the predetermined table 1060 in which the subtraction result values 1030 correspond to the indices 1050 related to the characteristic of the recycled ABS with respect to the distribution similarities 1040. The server 110 may select the index 1050 related to the characteristic of the recycled ABS and corresponding to the subtraction result value 1030 and the distribution similarity 1040 from the predetermined table 1060. Here, the distribution similarities 1040 have been described above, and the description will not be reiterated.

The present disclosure has been described with reference to various embodiments. It will be apparent to those of ordinary skill in the art that the present invention can be implemented in modified forms without departing from the essential characteristics of the present invention. Therefore, the disclosed embodiments should be interpreted as not being limiting but being illustrative. The scope of the present invention is defined not by the above descriptions but by the claims, and it should be understood that all differences within their equivalents are included in the present invention.

Meanwhile, the above-described embodiments of the present invention may be written as a program that is executable on a computer and implemented in a general-use digital computer that runs the program using a computer-readable recording medium. The computer-readable recording medium includes storage media such as magnetic storage media (e.g., a ROM, a floppy disk, and a hard disk) and optical storage media (e.g., a compact disc ROM (CD-ROM) and a digital versatile disc (DVD)).

1. An operating method of a system for estimating a property of a mixed material using recycled acrylonitrile butadiene styrene (ABS), which includes a server for analyzing data using a machine learning model, a user terminal for receiving an input of a user and transmitting the input to the server, and a data collection unit for collecting data, the operating method comprising:
performing, by the data collection unit, gel permeation chromatography (GPC) analysis on recycled ABS to acquire recycled ABS composition information including a recycled ABS-related number average molecular weight and a recycled ABS-related weight average molecular weight;
transmitting, by the data collection unit, the recycled ABS composition information to the server;
applying, by the server, the recycled ABS composition information to a first machine learning model to acquire an index related to a characteristic of the recycled ABS;
acquiring, by the user terminal, content ratios of the recycled ABS, general ABS, carbon nanotubes (CNT), carbon fiber (CF), and recycled thermoplastic polyurethane (TPU) included in a mixed material and expected property information of the mixed material;
transmitting, by the user terminal, the content ratios of the recycled ABS, the general ABS, the CNT, the CF, and the recycled TPU to the server;
applying, by the server, the index related to the characteristic of the recycled ABS and the content ratios of the recycled ABS, the general ABS, the CNT, the CF, and the recycled TPU to a second machine learning model to acquire an index related to the property of the mixed material of the recycled ABS, the general ABS, the CNT, the CF, and the recycled TPU and a reliability of the index;
transmitting, by the server, the index related to the property of the mixed material and the reliability of the index to the user terminal;
determining, by the user terminal, a range of the property of the mixed material corresponding to the index related to the property of the mixed material;
generating, by the user terminal, a first output signal representing that it is essentially necessary to perform an experiment when the reliability of the index is lower than a first threshold reliability; and
generating, by the user terminal, a second output signal representing that it is necessary to perform an additional experiment when the expected property information of the mixed material is within the range of the property of the mixed material and the reliability of the index is higher than a second threshold reliability,
wherein the first machine learning model is a model which has performed machine learning on a relationship between recycled ABS composition information and indices related to the characteristic of recycled ABS, and
the second machine learning model is a model which has performed machine learning on relationships between indices related to the property of mixed materials and the index related to the characteristic of the recycled ABS, content ratios of the recycled ABS, the general ABS, the CNT, the CF, and the recycled TPU.

2. The operating method of claim 1, comprising:
acquiring, by the server, a plurality of pieces of past recycled ABS composition information and indices related to the characteristic of a plurality of pieces of past recycled ABS each corresponding to the plurality of pieces of past recycled ABS composition information; and
performing, by the server, machine learning on relationships between the plurality of pieces of past recycled ABS composition information and the indices related to the characteristic of the plurality of pieces of past recycled ABS to generate the first machine learning model.

3. The operating method of claim 1, wherein the index related to the characteristic of the recycled ABS includes information related to a degree of breakage of polymer chains made of acrylonitrile, butadiene, and styrene included in the recycled ABS.

4. The operating method of claim 2, comprising:
acquiring, by the server, the indices related to the characteristic of the plurality of pieces of past recycled ABS, content ratios of the plurality of pieces of past recycled ABS, content ratios of a plurality of pieces of past general ABS, content ratios of a plurality of pieces of past CNT, content ratios of a plurality of pieces of past CF, content ratios of a plurality of pieces of past recycled TPU, and indices related to the property of a plurality of past mixed materials; and
performing, by the server, machine learning on the indices related to the characteristic of the plurality of pieces of past recycled ABS, the content ratios of the plurality of pieces of past recycled ABS, the content ratios of the plurality of pieces of past general ABS, the content ratios of the plurality of pieces of past CNT, the content ratios of the plurality of pieces of past CF, the content ratios of the plurality of pieces of past recycled TPU, and the indices related to the property of the plurality of past mixed materials to generate the second machine learning model.

5. The operating method of claim 1, wherein the acquiring of the recycled ABS composition information comprises:
performing GPC analysis on the recycled ABS to generate a distribution of molecular weights of molecules included in the recycled ABS;
performing GPC analysis on the general ABS to generate a distribution of molecular weights of molecules included in the general ABS; and
determining a similarity between the distribution of the molecular weights of the molecules included in the recycled ABS and the distribution of the molecular weights of the molecules included in the general ABS,
wherein the transmitting of the recycled ABS composition information to the server comprises transmitting the similarity between the distributions to the server, and
wherein the acquiring of the index related to the characteristic of the recycled ABS comprises acquiring, by the server, an index related to the characteristic of the recycled ABS on the basis of the similarity between the distributions without using the first machine learning model when the similarity between the distributions is a first threshold similarity or more.

6. The operating method of claim 5, wherein the determining of the similarity comprises:
determining a first molecular weight (A2) having a maximum number (A1) in the distribution of the molecular weights of the molecules included in the recycled ABS;
determining a second molecular weight (B2) having a maximum number (B1) in the distribution of the molecular weights of the molecules included in the general ABS; and
determining the similarity between the distributions as follows: the similarity between the distributions=1/((((A2−B2)\hat{ }2)/(A2\hat{ }A2+B2\hat{ }B2))\hat{ }1/2)+(((A1−B1)\hat{ }2)/(A1\hat{ }2+B1\hat{ }2))\hat{ }(1/2)).

7. The operating method of claim 6, wherein the acquiring of the index related to the characteristic of the recycled ABS on the basis of the similarity comprises acquiring an index related to the characteristic of the recycled ABS to represent a lowest degree of breakage of polymer chains made of acrylonitrile, butadiene, and styrene included in the recycled ABS when the similarity between the distributions is the first threshold similarity or more.

8. The operating method of claim 7, wherein the content ratio of the general ABS is one or less which is a ratio of a mass of the general ABS to a mass of the recycled ABS,
the content ratio of the CNT is 1/20 or less which is a ratio of a mass of the CNT to the mass of the recycled ABS,
the content ratio of the CF is 1/5 or less which is a ratio of a mass of the CF to the mass of the recycled ABS, and
the content ratio of the recycled TPU is 2/3 or less which is a ratio of a mass of the recycled TPU to the mass of the recycled ABS.
